(19) United States
(12) Patent Application Publication
(10) Pub. No.: US 2009/0068219 A1
(43) Pub. Date: Mar. 12, 2009

(54) Anti-wrinkle hormone-type cosmetic composition

(75) Inventors: Michelle Elie, New York, NY (US); John Kressaty, Nyack, NY (US)

Correspondence Address: COHEN, PONTANI, LIEBERMAN & PAVANE LLP, 551 FIFTH AVENUE, SUITE 1210, NEW YORK, NY, 10176, US

(73) Assignee: 3LAB, Inc., Englewood, NJ (US)

(21) Appl. No.: 11/900,179

(22) Filed: Sep. 10, 2007

(51) Int. Cl. A61K 8/64; A61K 8/97; A61Q 19/08

(52) U.S. Cl. 424/195.16; 514/2

(57) ABSTRACT

A cosmetic anti-aging composition comprising an effective amount of Human Oligopeptide-9 in combination with at least one additional oligopeptide is provided. The composition displays a very surprisingly and highly beneficial synergistic effect for treatment of fine line and wrinkles, skin brightening, skin re-texturization, and/or thickening of the skin, etc.

### BACKGROUND OF THE INVENTION

1. Field of the Invention

The present invention relates to cosmetic compositions, in particular, anti-aging skin care products.

2. Description of the Related Art

Many skin care, anti-aging products are based upon a variety of technical approaches that reflect the current scientific thinking. Multiple combinations of materials whose functionality and efficacy contribute to the formulation of a successful treatment product are being utilized. Among these ingredients are MMP inhibitors (stop matrix metalloproteinases such as Collagenase and Elastase from breaking down the connective fibers of the extra-cellular matrix like Collagen and Elastin), oligopeptides of very diverse nature, and activators of specific skin enzymes. Combinations of these materials in many forms are nowadays used in the skin care market.

Nevertheless, until now, the use of a Human Oligopeptide mimicking and eliciting the same response as that of Human Growth Hormone (HGH), designed specifically for cosmetic products in synergistic combination with other selected oligopeptides, skin brightening agents, antioxidants (free radical scavengers) and function-specific botanical extracts of superior performance has not been proposed.

### SUMMARY OF THE INVENTION

Therefore, in accordance with one embodiment of the present invention, we provide a cosmetic anti-aging composition comprising an effective amount of Human Oligopeptide-9 in combination with at least one additional oligopeptide.

The at least one additional oligopeptide may be of such a molecular weight that they are able to penetrate the skin. Without being bound by any theory, the at least one additional oligopeptide may send signal the skin to bio-synthesize more of the parent protein. For example, the at least one additional oligopeptitide may be Oligopeptide-4 and/or Oligopeptide-5. For example, Oligopepetide-5 (tradename Dermonectin—manufactured by Vevy Europe S.p.A., Italy) may send signal to the skin, leading to a synergistic efficacy between the matrix protein Fibronectin (parent protein of Oligopeptide-5) and the Human Growth Hormone (tradename Nanoclaire-GY, manufactured by Regeron, South Korea).

The amount of Human Oligopeptide-9 may be from about 0.001% to about 5%, preferably from about 0.001% to about 1%, based on the total weight of the cosmetic composition. The amount of at least one additional oligopeptide may be from about 0.001% to about 3%, preferably from 0.001% to about 1%, based on the total weight of the cosmetic composition. The ratio of Human Oligopeptide-9 and the at least one additional oligopeptide by weight may be in the range of about 1:1 to about 1:5.

In accordance with another embodiment of the present invention, the cosmetic composition may comprise an effective amount of Hydrolyzed Glycosaminoglycans (tradename Hyaluramine,manufactured by Vevy Europe S.p.A., Italy), Xylitol derivatives (tradename Aquaxyl, manufactured by Seppic, France) and a blend of Beet Root Extract, Haberlea Rhodopensis Leaf Extract and yeast Extract (tradename Unisurrection S-61, manufactured by Induchem, Switzerland). A cosmetic product containing these ingredients exhibits a surprisingly effective efficacy as an internal moisturizer or hydrator. These ingredients may ensure that moisture is always available to the skin from the inner layers to the Stratum Corneum in an outward gradient.

The amount of Hydrolyzed Glycosaminoglycans may be from about 0.1% to about 1% based on the total weight of the cosmetic composition. The amount of Xylitol derivatives may be from about 0.1% to about 3% based on the total weight of the cosmetic composition. The total amount of the blend mixture of Beet Root Extract, Haberlea Rhodopensis Leaf Extract and yeast Extract may be from about 0.1 to about 3% based on the total weight of the cosmetic composition.

The ratio of Hydrolyzed Glycosaminoglycans and the Xylitol derivatives by weight may be in the range of 1:1 to 1:5. The ratio of Hydrolyzed Glycosaminoglycans and the blend mixture of Beet Root Extract, Haberlea Rhodopensis Leaf Extract and yeast Extract by weight may be in the range of 1:1 to 1:5.

In addition to the above ingredients, the cosmetic composition may contain other cosmetically acceptable ingredients, such as skin brightening agents, antioxidants (free radical scavengers), function-specific botanical extracts, emulsion stabilizer/thickening agent, emulsifier, emollient, solvent, skin softener, and fragrance.

As a preferred embodiment, the cosmetic composition in accordance with the present invention may contain Water, Disodium EDTA, Xanthan Gum, Butylene Glycol, Propyl Gallate, Glycerin, Dimethyl Isosorbide, Hydroxyethylcellulose, Polymethylmethacrylate, C10-18 Triglyceride, Octyldodecyl Behenate, Neopentyl Glycol Dicaprate, PPG-3 Benzyl Ether Myristate, Dimethicone, C12-20 Acid PEG-8 Ester, Cetearyl Glucoside, Steareth-2, Tocopheryl Acetate, Hydroxyethyl Acrylate/Sodium Acryloyldimethyl Taurate Copolymer, Squalane, Polysorbate 60, Diacetyl Boldine, Lecithin, Human Oligopeptide-9, EDTA, Xylitylglucoside, Anhydroxylitol, Xylitol, Oligopeptide-4, Oligopeptide-5, Sodium Hyaluronate, Ginko Biloba Leaf Extract, Butylene Glycol, Beta Vulgaris (Beet) Root Extract, Yeast Extract, Camellia Sinensis (Green tea) Leaf Extract, Hydrolyzed Glycosaminoglycans, Glycerin, Sodium Levulinate, Citric Acid, and Fragrance.

The cosmetic composition in accordance with the present invention may be formulated in any suitable form such as serum, cream, lotion, and gel, preferably serum.

The composition of the present invention may be made following standard methods of preparation of oil in water emulsions in the laboratory. That is, the oil phase is prepared separately from the water phase and they are mixed at a specified temperature. Cooled and the labile ingredients are then added.

The final cosmetic composition in accordance with the present invention may have a pH range of about 4 to 7, preferably about 4 to 6, more preferably, from about 4.5 to 5.

It has been found been found that the cosmetic composition in accordance with the present invention displays a very surprisingly and highly beneficial synergistic effect for treatment of fine line and wrinkles, skin brightening, skin re-texturization, and/or thickening of the skin, etc.

Other objects and features of the present invention will become apparent from the following detailed description.

### DETAILED DESCRIPTION OF THE PRESENTLY PREFERRED EMBODIMENTS

The following examples further illustrate the present invention without limiting it.

### EXAMPLE 1

The following table shows a serum composition in accordance with the present invention.

TABLE 1

| Ingredient (TRADENAME) | INCI | % Weight |
|---|---|---|
| DEIONIZED WATER | Water | 68.475 |
| DISODIUM EDTA | Disodium EDTA | 0.050 |
| KELTROL CG | Xanthan Gum | 0.100 |
| BUTYLENE GLYCOL | Butylene Glycol | 2.000 |
| PROPYL GALLATE | Propyl Gallate | 0.100 |
| GLYCERIN | Glycerin | 2.000 |
| ARLASOLV DMI | Dimethyl Isosorbide | 3.000 |
| LIPORAMNOSAN | Hydroxyethylcellulose | 0.300 |
| MICROMA 100 | Polymethylmethacrylate | 0.500 |
| NESATOL | C10-18 Triglyceride | 1.500 |
| DERMOL 2022 | Octyldodecyl Behenate | 0.650 |
| DERMOL NGDC | Neopentyl Glycol Dicaprate | 3.000 |
| CRODAMOL STS | PPG-3 Benzyl Ether Myristate | 1.000 |
| DC 200/100CS | Dimethicone | 0.500 |
| XALIFIN 15 | C12-20 Acid PEG-8 Ester | 1.500 |
| MONTANOV 68 | Cetearyl Alcohol (and) Cetearyl Glucoside | 0.500 |
| PROCOL SA-2 | Steareth-2 | 0.125 |
| VITAMIN E ACETATE | Tocopheryl Acetate | 0.150 |
| SIMULGEL NS | Hydroxyethyl Acrylate/Sodium Acryloyldimethyl Taurate | 1.500 |
| | Copolymer (and) Squalane (and) Polysorbate 60 | |
| LUMISKIN | Diacetyl Boldine | 0.300 |
| EMBLICA | Phyllanthus Emblica Fruit Extract | 0.100 |
| NANOCLAIRE-GY | Lecithin (and) Human Oligopeptide-9 (and) Sodium Phosphate | 5.000 |
| | (and) Sodium Chloride (and) EDTA | |
| AQUAXYL | Xylitylgoucoside(and) Anhydroxylitol (and) Xylitol | 0.500 |
| COLLAGENON | Oligopeptide-4 | 2.000 |
| DERMONECTIN | Oligopeptide-5 | 1.000 |
| RITA HA 1-C | Sodium Hyaluronate | 1.000 |
| GINKO BILOBA EXTRACT BG | Ginko Biloba Leaf Extract (and) Butylene Glycol (and) Water | 0.100 |
| UNISURRECTION S-61 | Beta Vulgaris (Beet) Root Extract (and) Glycerin/Haberlea | 0.500 |
| | Rhodopensis Leaf Extract (and) Yeast Extract | |
| GREEN TEA EXTRACT | Camellia Sinensis(Green tea) Leaf Extract (and) Butylene | 0.100 |
| | Glycol (and) Water | |
| HYALURAMINE | Hydrolyzed Glycosaminoglycans | 0.150 |
| DERMOSOFT 1388 | Water (and) Glycerin (and) Sodium Levulinate (and) Sodium | 2.000 |
| | Anisate | |
| CITRIC ACID 50% SOLUTION | Citric Acid | 0.100 |
| MASKING AGENT 886621 | Fragrance | 0.100 |
| FRAGRANCE | | |
| UJ003775/00/PURE WHITE | Fragrance | 0.100 |
| Total | | 100 |

### EXAMPLE 2

The following example studies efficacy of a serum composition in accordance with one embodiment of the present invention.

### Multifaceted Photoaging Improvement Study

Ref. No.: MULTIFACET.M06-2.ENL

### 1.0 Objective:

To evaluate the efficacy of topical treatments applied on the face and eye area for a period of eight weeks to improve photoaging on the skin. Efficacy was measured with Spectrophotometer, Cutometer, Corneometer, eye area replicas, photography and a Self-assessment Questionnaire.

### 2.0 Test Material Handling:

| Lab No. | Sample Description |
|---|---|
| M06-2 | H Serum |
| M06-2A | Night Cream |
| M06-3 | 3Lab Sunblock |

### 2.1 Handling:

Each test material is assigned a unique laboratory code number and entered into a daily log identifying the lot number, sample description, sponsor, date received and tests requested.
Samples are retained for a period of three months beyond submission of final report unless otherwise specified by the sponsor or if sample is known to be in support of governmental applications, in which case retained samples are kept two years beyond final report submission.
Sample disposition is conducted in compliance with appropriate federal, state and local ordinances.

### 3.0 Population Demographics:

| Number of subjects enrolled | | 18 |
|---|---|---|
| Number of subjects completing study | | 16 |
| Age Range | | 36-63 |
| Sex | Female | 18 |
| Race | Caucasian | 17 |
| | Hispanic | 0 |
| | Asian | 1 |

### 3.1 Standards for Inclusion in a Study:

1. Individuals diagnosed by the investigator as having moderate photoaging with uneven pigmentation.
2. Females between the ages of 35 and 65
3. Individuals willing to discontinue all photoaging and related skin care products.
4. Individuals who have completed a preliminary medical history.
5. Individuals, who have read, understood and signed an informed consent document relating to the specific type of study to which they are subscribing. Consent forms are kept on file.
6. Individuals who understand the instructions for use and are willing to cooperate with the program as stated.
7. Individuals free of any dermatological or systemic disorder, or any appearance issue that may interfere with the accurate evaluation and/or results during the course of the study, at the discretion of the Investigator.
8. Individuals free of any acute or chronic disease that might interfere with or increase the risk of study participation.
9. Individuals able to cooperate with the Investigator and the research staff, are willing to have test materials applied according to protocol, and complete the full course of the study.
   3.2 Standards for Exclusion from a Study:
1. Individuals who are under doctor's care.
2. Individuals who are currently taking any medication that may mask or interfere with the test results.
3. Subjects with a history of any form of skin cancer, melanoma, lupus, psoriasis, connective tissue disease, diabetes, chronic skin allergies or any disease that would increase the risk associated with study participation.
4. Individuals who have dermatological disorder or personal appearance issue, which in the opinion of the Investigator would interfere with the accurate evaluation of the individual's face.
5. Individuals with known hypersensitivity to cosmetic products or with any history of sensitivity to cosmetics in general.
6. Individuals who are currently taking medication (topical or oral) which in the opinion of the Investigator would mask or interfere with the results.
7. Individuals who have had any surgical treatment performed on the facial area which will interfere with the test.
8. Individuals who are unwilling or unable to comply with the listed requirements especially discontinuation of all prescription or OTC cosmetic preparations to the face.
9. Individuals who have participated in another clinical trial or experimental drug within the past 30 days.
10. Female volunteers who indicate that they are pregnant, nursing, or planning a pregnancy.

### 4.0 Informed Consent:

A signed informed consent, as required by CFR Title 21, Part 50, was obtained from each panelist prior to initiating the study describing reasons for the study, possible adverse effects, associated risks and potential benefits of the treatment and their limits and liability. Each subject was assigned a permanent identification number and completed an extensive medical history form.

### 5.0 Institutional Ethics Committee (IEC):

The Independent Ethics Committee of Investigator consists of 5 or more individuals chosen from within the Investigator company for technical expertise and from the local community. The list of IEC members are kept on file.

### 6.0 Methodology:

KONICA MINOLTA SPECTROPHOTOMETER CM-2600d
This instrument utilizes the D/8 geometry conforming to CIE No. 15, ISO 7724/1, ASTM E1164, DIN 5033 Tei17, and JIS Z8722-1982 (diffused illumination/8° viewing system) standards, and offers simultaneous SCI (Specular Component Included) and SCE (Specular Component Excluded) measurements. Light from Xenon lamps diffuses on the inner surface of the integrating sphere and illuminates the specimen uniformly. The light reflected by the specimen surface at an angle of 8 degrees to the normal of the surface is received by the specimen-measuring optical system. The diffused light in the integrating sphere is received by the illumination-monitoring optical system and guided to the sensor. The light reflected by the specimen surface and the diffused light are divided into each wavelength component by the specimen-measuring optical system and illumination-monitoring optical sensor, respectively, and then signal proportional to the light intensity of each component is output to the analog processing circuit. By using the outputs from the specimen-measuring optical system and the illumination-monitoring sensor for calculation, compensation for slight fluctuation in spectral characteristics and the intensity of the illumination light is performed. (Double-beam system)
COURAGE+KHAZAKA CUTOMETER MPA 580
The Cutometer was used to measure the elasticity of the skin surface, using the vacuum principle. This measurement principle is based on suction and elongation. The probe sucks up a defined area of skin surface and records it optically. Analysis of the recorded measurement curves makes it possible to determine the elastic and plastic characteristics of the skin; viscoelasticity. Young skin shows a high degree of elasticity and loses shape only gradually while regaining its original state after the end of the suction procedure. Skin which is healthy, supple and adequately moist will have a higher elasticity than a dry, rough skin. The Cutometer therefore gives a set of measurements which allows us to quantify elastic characteristics.
SKIN SURFACE REPLICA
Skin surface impressions (replicas) were obtained from the crow's feet area of the face at day 0, week 4, and week 8 of product use. The coded skin surface replica specimens were analyzed using Image-Pro Plus software. One can measure changes in skin surface topography by selecting a gray level threshold that allows one to directly determine the projected area of the shadowed region associated with the wrinkles. This parameter, called Shadows, is expressed as a percent of the total area covered by the shadowing. If the surface is rather smooth and flat, there will be few shadows and this value will be small, but if the surface is wrinkled and rough, the shadowed areas will correspondingly increase. In addition, Ra is also computed, which involves generating an average line through the center of the profile and determining the area of deviation above and below this line
PHOTO BOOTH
Photographs were conducted using a photo booth with a 3-point head restraint with photographs taken of the frontal view, 45 degrees to the right, and 45 degrees to the left. The standard chin rest and a three-point adjustable head support ensure proper positioning of the panelist for each time point. Digital Photographs of the face were taken using Nikon Coolpix 8400 Digital Camera at Day 0 (pre-application), four weeks and eight weeks of product usage.
SELF-ASSESSMENT QUESTIONNAIRE
Panelists were asked to answer a consumer questionnaire, developed by the sponsor, at four weeks and eight weeks based on their experience with the test product. Questions were based on whether or not an improvement was noticed with fine lines/ wrinkles, roughness/dryness, appearance of age spots/freckles/skin discolorations, skin lightening, softness/smoothness, radiance/tone/clarity, firmness/tightness/elasticity, skin moisture, and overall appearance. Answers consisted of strongly agree, somewhat agree, somewhat disagree and strongly disagree.

### 7.0 Study Design:

Eighteen healthy females between the ages of 36 and 63 were inducted into this study. The panelists applied H Serum twice a day, morning and night, to cleansed skin as directed for the entire treatment period of the study (8 weeks). They were asked to use 3Lab Sunblock after morning application before going out. In addition, a Night Cream was applied before going to bed at night for the duration of the study.
At the baseline visit (day 0), all panelists completed the informed consent and medical history forms. Corneometer, Spectrophotometer, Cutometer measurements, photoggraphs and replicas were taken at baseline, week 4, and week 8. The panelists washed their face 30 minutes prior to making the replica to ensure that no make-up, oils or creams were on the skin while obtaining the replica and were asked to remain relaxed and free of any facial expressions in order to prevent alteration in the appearance of the crow's feet area. Panelists were also asked to assess product performance at 4 and 8 weeks of product use by completing a questionnaire designated to evaluate panelist's face for fine lines/wrinkles, roughness/dryness, appearance of age spots/freckles/skin discolorations, skin lightening, softness/smoothness, radiance/tone/clarity, firmness/tightness/elasticity, skin moisture, and overall appearance.
At the completion of the study, all unused study products were collected from the panelists and any adverse events during the study were recorded.

### 8.0 Results:

See attached tables.

### 9.0 Observations:

No adverse effects or unexpected reactions of any kind were observed on any of the subjects.

### 10.0 Conclusions:

Within the limits imposed by the conduct and population size of the study described herein, the test material (Lab No.: M06-2, Client No.: H Serum) demonstrated a significant improvement in the appearance of facial photoaging.

TABLE 2
SPECTROPHOTOMETER READINGS (Day 0, Week 4 & Week 8)
L values (SCI) of Hyper-Pigmented Spot
Lab Nos.: M06-2/M06-2A/M06-3
Client No.: H Serum/Night Cream/3Lab Sunblock

| No. | Panelist No. | Age | Race | Day 0 | Week 4 | % Diff | Week 8 | % Diff |
|---|---|---|---|---|---|---|---|---|
| 1 | M 219 | 59 | C | 51.26 | 52.15 | 1.73 | 51.59 | 0.64 |
| 2 | M 220 | 42 | C | 51.00 | 48.62 | −4.66 | 48.89 | −4.12 |
| 3 | M 221 | 53 | M | 46.51 | 48.06 | 3.33 | 48.47 | 4.22 |
| 4 | M 222 | 49 | C | 55.31 | 55.63 | 0.59 | 55.05 | −0.46 |
| 5 | M 224 | 36 | C | 58.55 | 58.54 | 0.00 | 59.57 | 1.76 |
| 6 | M 226 | 51 | C | 54.52 | 56.74 | 4.07 | 55.95 | 2.63 |
| 7 | M 227 | 54 | C | 48.64 | 48.80 | 0.34 | 50.98 | 4.82 |
| 8 | M 228 | 61 | C | 52.92 | 52.97 | 0.08 | 53.42 | 0.94 |
| 9 | M 229 | 44 | C | 55.67 | 55.99 | 0.59 | 57.35 | 3.03 |
| 10 | M 230 | 63 | C | 55.33 | 55.72 | 0.71 | 57.88 | 4.62 |
| 11 | M 231 | 45 | A | 52.36 | 54.46 | 4.02 | 57.62 | 10.06 |
| 12 | M 232 | 43 | C | 55.36 | 54.13 | −2.22 | 55.47 | 0.20 |
| 13 | M 233 | 55 | C | 49.81 | 52.24 | 4.88 | 52.92 | 6.23 |
| 14 | M 234 | 59 | C | 56.53 | 58.30 | 3.13 | 60.54 | 7.10 |
| 15 | M 235 | 53 | C | 50.11 | 51.79 | 3.35 | 50.74 | 1.25 |
| 16 | M 236 | 49 | C | 59.43 | 61.52 | 3.51 | 61.07 | 2.77 |

| | | | | | |
|---|---|---|---|---|---|
| MEAN | 53.33 | 54.10 | 1.47 | 54.84 | 2.85 |
| % DIFF | | 1.45 | | 2.84 | |
| t-Stat | | −2.31 | | −3.43 | |
| Statistical Significance | | Significant | | Significant | |

*Statistically Significant Critical Value = 1.7530

TABLE 3
SPECTROPHOTOMETER READINGS (Day 0, Week 4 & Week 8)
L values (SCE) of Hyper-Pigmented Spot
Lab Nos.: M06-2/M06-2A/M06-3
Client No.: H Serum/Night Cream/3Lab Sunblock

| No. | Panelist No. | Age | Race | Day 0 | Week 4 | % Diff | Week 8 | % Diff |
|---|---|---|---|---|---|---|---|---|
| 1 | M 219 | 59 | C | 50.75 | 52.02 | 2.51 | 51.50 | 1.49 |
| 2 | M 220 | 42 | C | 51.00 | 48.36 | −5.16 | 48.75 | −4.39 |
| 3 | M 221 | 53 | M | 46.33 | 47.93 | 3.45 | 48.37 | 4.41 |
| 4 | M 222 | 49 | C | 55.20 | 55.58 | 0.71 | 54.90 | −0.53 |
| 5 | M 224 | 36 | C | 58.45 | 58.36 | −0.14 | 59.43 | 1.70 |
| 6 | M 226 | 51 | C | 54.41 | 56.59 | 4.01 | 55.72 | 2.42 |
| 7 | M 227 | 54 | C | 48.51 | 48.54 | 0.07 | 50.59 | 4.31 |
| 8 | M 228 | 61 | C | 52.69 | 52.92 | 0.44 | 53.30 | 1.14 |
| 9 | M 229 | 44 | C | 55.66 | 55.87 | 0.38 | 57.23 | 2.82 |
| 10 | M 230 | 63 | C | 55.26 | 55.81 | 1.00 | 57.99 | 4.94 |
| 11 | M 231 | 45 | A | 52.16 | 54.12 | 3.75 | 57.17 | 9.61 |
| 12 | M 232 | 43 | C | 55.27 | 53.98 | −2.33 | 55.33 | 0.11 |
| 13 | M 233 | 55 | C | 49.63 | 52.01 | 4.81 | 52.79 | 6.39 |
| 14 | M 234 | 59 | C | 56.43 | 58.24 | 3.20 | 60.40 | 7.04 |
| 15 | M 235 | 53 | C | 49.88 | 51.76 | 3.78 | 50.63 | 1.50 |
| 16 | M 236 | 49 | C | 59.33 | 61.39 | 3.47 | 60.93 | 2.69 |

| MEAN | 53.19 | 53.97 | 1.33 | 54.69 | 2.85 |
|---|---|---|---|---|---|
| % DIFF | | 1.47 | | 2.83 | |
| t-Stat | | −2.26 | | −3.48 | |
| Statistical Significance | | Significant | | Significant | |

*Statistically Significant Critical Value = 1.7530

TABLE 4
SPECTROPHOTOMETER READINGS (Day 0, Week 4 & Week 8)
L values (SCI) of Surrounding Skin
Lab Nos.: M06-2/M06-2A/ M06-3
Client No.: H Serum/Night Cream/3Lab Sunblock

| No. | Panelist No. | Age | Race | Day 0 | Week 4 | % Diff | Week 8 | % Diff |
|---|---|---|---|---|---|---|---|---|
| 1 | M 219 | 59 | C | 64.00 | 64.50 | 0.79 | 63.28 | −1.12 |
| 2 | M 220 | 42 | C | 55.70 | 54.80 | −1.62 | 54.10 | −2.87 |
| 3 | M 221 | 53 | C | 55.35 | 56.25 | 1.64 | 55.78 | 0.80 |
| 4 | M 222 | 49 | C | 60.20 | 59.10 | −1.83 | 60.27 | 0.12 |
| 6 | M 224 | 36 | C | 61.03 | 60.87 | −0.27 | 61.04 | 0.02 |
| 8 | M 226 | 51 | C | 62.35 | 62.80 | 0.72 | 62.39 | 0.07 |
| 9 | M 227 | 54 | C | 61.54 | 52.25 | −15.09 | 61.70 | 0.28 |
| 10 | M 228 | 61 | C | 60.86 | 60.80 | −0.09 | 61.97 | 1.84 |
| 11 | M 229 | 44 | C | 60.92 | 62.36 | 2.36 | 61.92 | 1.63 |
| 12 | M 230 | 63 | C | 65.82 | 66.57 | 1.13 | 65.73 | −0.15 |
| 13 | M 231 | 45 | A | 59.37 | 60.96 | 2.68 | 61.93 | 4.30 |
| 14 | M 232 | 43 | C | 61.14 | 62.62 | 2.44 | 61.32 | 0.33 |
| 15 | M 233 | 55 | C | 62.31 | 63.54 | 1.98 | 64.30 | 3.21 |
| 16 | M 234 | 59 | C | 63.93 | 62.12 | −2.82 | 62.98 | −1.48 |
| 17 | M 235 | 53 | C | 63.27 | 62.73 | −0.85 | 63.12 | −0.25 |
| 18 | M 236 | 49 | C | 65.42 | 66.82 | 2.14 | 66.70 | 1.95 |

| | | | | | |
|---|---|---|---|---|---|
| MEAN | 61.45 | 61.19 | −0.42 | 61.78 | 0.54 |
| % DIFF | | −0.42 | | 0.54 | |
| t-Stat | | 0.39 | | −1.25 | |
| Statistical Significance | | Significant | | Significant | |

*Statistically Significant Critical Value = 1.7530

TABLE 5
SPECTROPHOTOMETER READINGS (Day 0. Week 4 & Week 8)
L values (SCE) of Surrounding Skin
Lab Nos.: M06-2/M06-2A/M06-3
Client No.: H Serum/Night Cream/3Lab Sunblock

| | | | | | | | | |
|---|---|---|---|---|---|---|---|---|
| No. | Panelist No. | Age | Race | Day 0 | Week 4 | % Diff | Week 8 | % Diff |

| | | | | | | | | |
|---|---|---|---|---|---|---|---|---|
| 1 | M 219 | 59 | C | 64.02 | 64.42 | 0.64 | 63.22 | −1.24 |
| 2 | M 220 | 42 | C | 55.68 | 54.69 | −1.78 | 54.03 | −2.97 |
| 3 | M 221 | 53 | C | 55.17 | 56.10 | 1.70 | 55.66 | 0.91 |
| 4 | M 222 | 49 | C | 59.97 | 58.97 | −1.67 | 60.10 | 0.21 |
| 5 | M 224 | 36 | C | 60.90 | 60.73 | −0.29 | 60.88 | −0.03 |
| 6 | M 226 | 51 | C | 62.21 | 62.63 | 0.67 | 62.19 | −0.03 |
| 7 | M 227 | 54 | C | 61.34 | 52.26 | −14.80 | 61.54 | 0.34 |
| 8 | M 228 | 61 | C | 60.75 | 60.67 | −0.13 | 61.88 | 1.87 |
| 9 | M 229 | 44 | C | 60.82 | 62.14 | 2.17 | 61.71 | 1.47 |
| 10 | M 230 | 63 | C | 65.66 | 66.52 | 1.31 | 65.64 | −0.03 |
| 11 | M 231 | 45 | A | 58.96 | 60.48 | 2.58 | 61.32 | 4.00 |
| 12 | M 232 | 43 | C | 61.14 | 62.48 | 2.20 | 61.23 | 0.17 |
| 13 | M 233 | 55 | C | 62.06 | 63.46 | 2.27 | 64.22 | 3.49 |
| 14 | M 234 | 59 | C | 63.78 | 61.91 | −2.94 | 62.82 | −1.50 |
| 15 | M 235 | 53 | C | 63.16 | 62.51 | −1.03 | 62.91 | −0.40 |
| 16 | M 236 | 49 | C | 65.39 | 66.74 | 2.07 | 66.61 | 1.87 |

| | | | | | |
|---|---|---|---|---|---|
| MEAN | 61.31 | 61.04 | −0.39 | 61.62 | 0.45 |
| % DIFF | | −0.44 | | 0.51 | |
| t-Stat | | 0.42 | | −1.16 | |
| Statistical Significance | | Significant | | Significant | |

*Statistically Significant Critical Value = 1.7530

TABLE 6
CUTOMETER READINGS (Day 0, Week 4 & Week 8)
R2 Gross Elasticity
Lab Nos.: M06-2/M06-2A/M06-3
Client No.: H Serum/Night Cream/3Lab Sunblock

| No. | Panelist No. | Age | Race | Day 0 | Week 4 | % Diff | Week 8 | % Diff |
|---|---|---|---|---|---|---|---|---|
| 1 | M 219 | 59 | C | 0.5874 | 0.5278 | −10.15 | 0.6538 | 11.30 |
| 2 | M 220 | 42 | C | 0.4830 | 0.5333 | 10.41 | 0.7692 | 59.25 |
| 3 | M 221 | 53 | C | 0.4459 | 0.6374 | 42.95 | 0.5224 | 17.16 |
| 4 | M 222 | 49 | C | 0.3826 | 0.4945 | 29.25 | 0.4881 | 27.57 |
| 5 | M 224 | 36 | C | 0.4880 | 0.6832 | 40.00 | 0.6286 | 28.81 |
| 6 | M 226 | 51 | C | 0.6777 | 0.6850 | 1.08 | 0.7436 | 9.72 |
| 7 | M 227 | 54 | C | 0.4328 | 0.5862 | 35.44 | 0.4725 | 9.17 |
| 8 | M 228 | 61 | C | 0.6519 | 0.5682 | −12.84 | 0.7207 | 10.55 |
| 9 | M 229 | 44 | C | 0.6190 | 0.5735 | −7.35 | 0.5000 | −19.22 |
| 10 | M 230 | 63 | C | 0.6182 | 0.4750 | −23.16 | 0.5213 | −15.67 |
| 11 | M 231 | 45 | A | 0.5476 | 0.6286 | 14.79 | 0.6053 | 10.54 |
| 12 | M 232 | 43 | C | 0.7069 | 0.6750 | −4.51 | 0.6241 | −11.71 |
| 13 | M 233 | 55 | C | 0.5231 | 0.6000 | 14.70 | 0.6098 | 16.57 |
| 14 | M 234 | 59 | C | 0.5688 | 0.5591 | −1.71 | 0.7342 | 29.08 |
| 15 | M 235 | 53 | C | 0.6064 | 0.6167 | 1.70 | 0.6500 | 7.19 |
| 16 | M 236 | 49 | C | 0.5944 | 0.6731 | 13.24 | 0.6000 | 0.94 |

| | | | | | |
|---|---|---|---|---|---|
| MEAN | 0.5584 | 0.5948 | 8.99 | 0.6152 | 11.95 |
| % DIFF | | 6.52 | | 10.19 | |
| t-Stat | | −1.48 | | −2.26 | |
| Statistical Significance | | Significant | | Significant | |

*Statistically Significant Critical Value = 1.7530

TABLE 7
CUTOMETER READINGS (Day 0, Week 4 & Week 8)
R5-Net Elasticity
Lab Nos.: M06-2/M06-2A/M06-3
Client No.: H Serum/Night Cream/3Lab Sunblock

| No. | Panelist No. | Age | Race | Day 0 | Week 4 | % Diff | Week 8 | % Diff |
|---|---|---|---|---|---|---|---|---|
| 1 | M 219 | 59 | C | 0.4731 | 0.5000 | 5.69 | 0.5778 | 22.13 |
| 2 | M 220 | 42 | C | 0.3608 | 0.4030 | 11.70 | 0.7292 | 102.11 |
| 3 | M 221 | 53 | C | 0.3684 | 0.5849 | 58.77 | 0.4722 | 28.18 |
| 4 | M 222 | 49 | C | 0.4000 | 0.375 | −6.25 | 0.4792 | 19.80 |
| 5 | M 224 | 36 | C | 0.3896 | 0.6364 | 63.35 | 0.5500 | 41.17 |
| 6 | M 226 | 51 | C | 0.6842 | 0.5 | −26.92 | 0.7556 | 10.44 |
| 7 | M 227 | 54 | C | 0.4110 | 0.5 | 21.65 | 0.4074 | −0.88 |
| 8 | M 228 | 61 | C | 0.3730 | 0.3977 | 6.62 | 0.5652 | 51.53 |
| 9 | M 229 | 44 | C | 0.5283 | 0.5366 | 1.57 | 0.3871 | −26.73 |
| 10 | M 230 | 63 | C | 0.5846 | 0.3974 | −32.02 | 0.4423 | −24.34 |
| 11 | M 231 | 45 | A | 0.4615 | 0.5476 | 18.66 | 0.6111 | 32.42 |
| 12 | M 232 | 43 | C | 0.5808 | 0.5096 | −12.26 | 0.5000 | −13.91 |
| 13 | M 233 | 55 | C | 0.3953 | 0.4902 | 24.01 | 0.6739 | 70.48 |
| 14 | M 234 | 59 | C | 0.5077 | 0.4167 | −17.92 | 0.7317 | 44.12 |
| 15 | M 235 | 53 | C | 0.5574 | 0.4750 | −14.78 | 0.8182 | 46.79 |
| 16 | M 236 | 49 | C | 0.5325 | 0.4348 | −18.35 | 0.4521 | −15.10 |

| | | | | | |
|---|---|---|---|---|---|
| MEAN | 0.4755 | 0.4816 | 5.22 | 0.5721 | 24.26 |
| % DIFF | | 1.27 | | 20.30 | |
| t-Stat | | −0.19 | | −2.51 | |
| Statistical Significance | | Significant | | Significant | |

*Statistically Significant Critical Value = 1.7530

TABLE 8
CUTOMETER READINGS (Day 0, Week 4 & Week 8)
R6-Viscoelasticity
Lab Nos.: M06-2/M06-2A/M06-3
Client No. H Serum/Night Cream/3Lab Sunblock

| No. | Panelist No. | Age | Race | Day 0 | Week 4 | % Diff | Week 8 | % Diff |
|---|---|---|---|---|---|---|---|---|
| 1 | M 219 | 59 | C | 0.5376 | 0.7143 | 32.87 | 0.7333 | 36.40 |
| 2 | M 220 | 42 | C | 0.5155 | 0.5672 | 10.03 | 0.8958 | 73.77 |
| 3 | M 221 | 53 | C | 0.6526 | 0.7170 | 9.87 | 0.8611 | 31.95 |
| 4 | M 222 | 49 | C | 0.8625 | 0.6250 | −27.54 | 0.7500 | −13.04 |
| 5 | M 224 | 36 | C | 0.6234 | 0.5303 | −14.93 | 0.7500 | 20.31 |
| 6 | M 226 | 51 | C | 0.5921 | 0.4432 | −25.15 | 0.7333 | 23.85 |
| 7 | M 227 | 54 | C | 0.8356 | 0.5676 | −32.07 | 0.6852 | −18.00 |
| 8 | M 228 | 61 | C | 0.4365 | 0.5000 | 14.55 | 0.6087 | 39.45 |
| 9 | M 229 | 44 | C | 0.5849 | 0.6585 | 12.58 | 0.4194 | −28.30 |
| 10 | M 230 | 63 | C | 0.6923 | 0.5385 | −22.22 | 0.8077 | 16.67 |
| 11 | M 231 | 45 | A | 0.6154 | 0.6667 | 8.34 | 1.1111 | 80.55 |
| 12 | M 232 | 43 | C | 0.3892 | 0.5385 | 38.36 | 0.5114 | 31.40 |
| 13 | M 233 | 55 | C | 0.5116 | 0.5686 | 11.14 | 0.7826 | 52.97 |
| 14 | M 234 | 59 | C | 0.6769 | 0.5500 | −18.75 | 0.9268 | 36.92 |
| 15 | M 235 | 53 | C | 0.5410 | 0.5000 | −7.58 | 0.8182 | 51.24 |
| 16 | M 236 | 49 | C | 0.8571 | 0.5072 | −40.82 | 0.5753 | −32.88 |

| | | | | | |
|---|---|---|---|---|---|
| MEAN | 0.6203 | 0.5745 | −3.21 | 0.7481 | 25.20 |
| % DIFF | | −7.37 | | 20.61 | |
| t-Stat | | 1.17 | | −2.44 | |
| Statistical Significance | | Significant | | Significant | |

*Statistically Significant Critical Value = 1.7530

TABLE 9
CUTOMETER READINGS (Day 0, Week 4 & Week 8)
R7-Elasticity
Lab Nos.: M06-2/M06-2A/M06-3
Client No.: H Serum/Night Cream/3Lab Sunblock

| No. | Panelist No. | Age | Race | Day 0 | Week 4 | % Diff | Week 8 | % Diff |
|---|---|---|---|---|---|---|---|---|
| 1 | M 219 | 59 | C | 0.3077 | 0.2917 | −5.20 | 0.3333 | 8.32 |
| 2 | M 220 | 42 | C | 0.2381 | 0.2571 | 7.98 | 0.3846 | 61.53 |
| 3 | M 221 | 53 | C | 0.2229 | 0.3407 | 52.85 | 0.2537 | 13.82 |
| 4 | M 222 | 49 | C | 0.2148 | 0.2308 | 7.45 | 0.2738 | 27.47 |
| 5 | M 224 | 36 | C | 0.2400 | 0.4158 | 73.25 | 0.3143 | 30.96 |
| 6 | M 226 | 51 | C | 0.4298 | 0.3465 | −19.38 | 0.4359 | 1.42 |
| 7 | M 227 | 54 | C | 0.2239 | 0.3190 | 42.47 | 0.2418 | 7.99 |
| 8 | M 228 | 61 | C | 0.2597 | 0.2652 | 2.12 | 0.3514 | 35.31 |
| 9 | M 229 | 44 | C | 0.3333 | 0.3235 | −2.94 | 0.2727 | −18.18 |
| 10 | M 230 | 63 | C | 0.3455 | 0.2583 | −25.24 | 0.2447 | −29.18 |
| 11 | M 231 | 45 | A | 0.2857 | 0.3286 | 15.02 | 0.2895 | 1.33 |
| 12 | M 232 | 43 | C | 0.4181 | 0.3313 | −20.76 | 0.3308 | −20.88 |
| 13 | M 233 | 55 | C | 0.2615 | 0.3125 | 19.50 | 0.3780 | 44.55 |
| 14 | M 234 | 59 | C | 0.3028 | 0.2688 | −11.23 | 0.3797 | 25.40 |
| 15 | M 235 | 53 | C | 0.3617 | 0.3167 | −12.44 | 0.4500 | 24.41 |
| 16 | M 236 | 49 | C | 0.2867 | 0.2885 | 0.63 | 0.2870 | 0.10 |

| | | | | | |
|---|---|---|---|---|---|
| MEAN | 0.2958 | 0.3059 | 7.75 | 0.3263 | 13.40 |
| % DIFF | | 3.44 | | 10.33 | |
| t-Stat | | −0.55 | | −1.73 | |
| Statistical Significance | | Significant | | Significant | |

*Statistically Significant Critical Value = 1.7530

TABLE 10
CUTOMETER READINGS (Day 0, Week 4 & Week 8)
F2-Logarithmical Average of the Maximum and Minimum Amplitudes
(Above the upper envelope-curve)
Lab Nos.: M06-2/M06-2A/M06-3
Client No.: H Serum/Night Cream/3Lab Sunblock

| No. | Panelist No. | Age | Race | Day 0 | Week 4 | % Diff | Week 8 | % Diff |
|---|---|---|---|---|---|---|---|---|
| 1 | M 219 | 59 | C | 0.2731 | 0.1523 | −44.23 | 0.1627 | −40.42 |
| 2 | M 220 | 42 | C | 0.3151 | 0.2453 | −22.15 | 0.3867 | 22.72 |
| 3 | M 221 | 53 | C | 0.2822 | 0.2707 | −4.08 | 0.1531 | −45.75 |
| 4 | M 222 | 49 | C | 0.3804 | 0.3385 | −11.01 | 0.272 | −28.50 |
| 5 | M 224 | 36 | C | 0.2967 | 0.2351 | −20.76 | 0.2309 | −22.18 |
| 6 | M 226 | 51 | C | 0.2433 | 0.3183 | 30.83 | 0.1952 | −19.77 |
| 7 | M 227 | 54 | C | 0.3175 | 0.2986 | −5.95 | 0.2256 | −28.94 |
| 8 | M 228 | 61 | C | 0.0000 | 0.2742 | — | 0.2774 | — |
| 9 | M 229 | 44 | C | 0.2053 | 0.1860 | −9.40 | 0.1590 | −22.55 |
| 10 | M 230 | 63 | C | 0.2231 | 0.4103 | 83.91 | 0.3041 | 36.31 |
| 11 | M 231 | 45 | A | 0.1942 | 0.1966 | 1.24 | 0.2633 | 35.58 |
| 12 | M 232 | 43 | C | 0.4342 | 0.5359 | 23.42 | 0.2313 | −46.73 |
| 13 | M 233 | 55 | C | 0.2852 | 0.1948 | −31.70 | 0.2845 | −0.25 |
| 14 | M 234 | 59 | C | 0.2553 | 0.1715 | −32.82 | 0.1840 | −27.93 |
| 15 | M 235 | 53 | C | 0.2145 | 0.1434 | −33.15 | 0.1484 | −30.82 |
| 16 | M 236 | 49 | C | 0.3266 | 0.1808 | −44.64 | 0.1802 | −44.83 |

| | | | | | |
|---|---|---|---|---|---|
| MEAN | 0.2654 | 0.2595 | −8.03 | 0.2287 | −17.60 |
| % DIFF | | −2.22 | | −13.85 | |
| t-Stat | | 0.21 | | 1.27 | |
| Statistical Significance | | Significant | | Significant | |

*Statistically Significant Critical Value = 1.7530

TABLE 11
ANALYSIS OF REPLICAS
RA North-South

| No. | Panelist No. | Age | Race | Day 0 | Week 4 | % Diff | Week 8 | % Diff |
|---|---|---|---|---|---|---|---|---|
| 1 | M 219 | 59 | C | 22.8 | 26.1 | 14.5 | 18.6 | −18.4 |
| 2 | M 220 | 42 | C | 16.9 | 14.1 | −16.6 | 17.3 | 2.4 |
| 3 | M 221 | 53 | C | 17.6 | 15.8 | −10.2 | 21.8 | 23.9 |
| 4 | M 222 | 49 | C | 29.1 | 20.2 | −30.6 | 23.5 | −19.2 |
| 5 | M 224 | 36 | C | 9.7 | 12.8 | 32.0 | 10.6 | 9.3 |
| 6 | M 227 | 54 | C | 26.3 | 17.2 | −34.6 | 12.0 | −54.4 |
| 7 | M 228 | 61 | C | 15.4 | 12.9 | −16.2 | 18.9 | 22.7 |
| 8 | M 229 | 44 | C | 8.3 | 12.8 | 54.2 | 14.1 | 69.9 |
| 9 | M 230 | 63 | C | 12.7 | 10.8 | −15.0 | 11.3 | −11.0 |
| 10 | M 231 | 45 | A | 16.7 | 12.7 | −24.0 | 12.2 | −26.9 |
| 11 | M 232 | 43 | C | 13.9 | 14.8 | 6.5 | 16.2 | 16.5 |
| 12 | M 233 | 55 | C | 15.1 | 8.7 | −42.4 | 13.8 | −8.6 |
| 13 | M 234 | 59 | C | 15.3 | 12.4 | −19.0 | 11.1 | −27.5 |
| 14 | M 236 | 49 | C | 11.0 | 11.7 | 6.4 | 13.2 | 20.0 |

| | | | | | |
|---|---|---|---|---|---|
| MEAN | 16.49 | 14.50 | −6.78 | 15.33 | −0.10 |
| % Diff | | −12.04 | | −7.02 | |
| t-Stat | | 1.75 | | 0.83 | |
| Statistical Significance | | Significant | | Significant | |

*Statistically Significant Critical Value = 1.7709
*M226 Data removed due to poor replica quality.
**M235 Data removed due to inconsistent facial expression.

TABLE 12
ANALYSIS OF REPLICAS
RA East-West

| No. | Panelist No. | Age | Race | Day 0 | Week 4 | % Diff | Week 8 | % Diff |
|---|---|---|---|---|---|---|---|---|
| 1 | M 219 | 59 | C | 13.0 | 17.2 | 32.3 | 13.1 | 0.8 |
| 2 | M 220 | 42 | C | 11.9 | 13.7 | 15.1 | 13.5 | 13.4 |
| 3 | M 221 | 53 | C | 17.4 | 15.3 | −12.1 | 18.9 | 8.6 |
| 4 | M 222 | 49 | C | 17.8 | 14.6 | −18.0 | 16.4 | −7.9 |
| 5 | M 224 | 36 | C | 13.4 | 13.4 | 0.0 | 12.6 | −6.0 |
| 6 | M 227 | 54 | C | 17.2 | 14.7 | −14.5 | 12.6 | -26.7 |
| 7 | M 228 | 61 | C | 14.2 | 9.8 | −31.0 | 18.0 | 26.8 |
| 8 | M 229 | 44 | C | 13.2 | 12.0 | −9.1 | 15.1 | 14.4 |
| 9 | M 230 | 63 | C | 13.5 | 10.3 | −23.7 | 11.6 | −14.1 |
| 10 | M 231 | 45 | A | 14.8 | 13.6 | −8.1 | 13.8 | −6.8 |
| 11 | M 232 | 43 | C | 16.5 | 12.5 | −24.2 | 13.4 | −18.8 |
| 12 | M 233 | 55 | C | 11.5 | 13.0 | 13.0 | 13.9 | 20.9 |
| 13 | M 234 | 59 | C | 11.9 | 12.8 | 7.6 | 9.4 | −21.0 |
| 14 | M 236 | 49 | C | 13.3 | 11.9 | −10.5 | 14.9 | 12.0 |

| | | | | | |
|---|---|---|---|---|---|
| MEAN | 14.26 | 13.20 | −5.94 | 14.09 | −0.31 |
| % Diff | | −7.41 | | −1.20 | |
| t-Stat | | 1.60 | | 0.27 | |
| Statistical Significance | | Significant | | Significant | |

*Statistically Significant Critical Value = 1.7709
*M226 Data removed due to poor replica quality.
**M235 Data removed due to inconsistent facial expression.

TABLE 13
ANALYSIS OF REPLICAS
Shadows North-South

| No. | Panelist No. | Age | Race | Day 0 | Week 4 | % Diff | Week 8 | % Diff |
|---|---|---|---|---|---|---|---|---|
| 1 | M 219 | 59 | C | 30.0 | 38.7 | 29.0 | 29.4 | −2.0 |
| 2 | M 220 | 42 | C | 24.3 | 21.0 | −13.6 | 25.7 | 5.8 |
| 3 | M 221 | 53 | C | 25.7 | 22.4 | −12.8 | 32.1 | 24.9 |
| 4 | M 222 | 49 | C | 40.5 | 32.0 | −21.0 | 35.5 | −12.3 |
| 5 | M 224 | 36 | C | 13.5 | 21.6 | 60.0 | 16.2 | 20.0 |
| 6 | M 227 | 54 | C | 42.7 | 35.6 | −16.6 | 22.8 | −46.6 |
| 7 | M 228 | 61 | C | 30.7 | 22.9 | −25.4 | 31.7 | 3.3 |
| 8 | M 229 | 44 | C | 15.8 | 31.9 | 101.9 | 28.6 | 81.0 |
| 9 | M 230 | 63 | C | 27.3 | 25.9 | −5.1 | 22.0 | −19.4 |
| 10 | M 231 | 45 | A | 32.5 | 30.1 | −7.4 | 25.0 | −23.1 |
| 11 | M 232 | 43 | C | 30.9 | 36.7 | 18.8 | 30.1 | −2.6 |
| 12 | M 233 | 55 | C | 28.8 | 25.9 | −10.1 | 31.6 | 9.7 |
| 13 | M 234 | 59 | C | 28.3 | 28.0 | −1.1 | 27.9 | −1.4 |
| 14 | M 236 | 49 | C | 40.1 | 34.6 | −13.7 | 32.3 | −19.5 |

| | | | | | |
|---|---|---|---|---|---|
| MEAN | 29.36 | 29.09 | 5.92 | 27.92 | 1.27 |
| % Diff | | −0.92 | | −4.91 | |
| t-Stat | | 0.14 | | 0.71 | |
| Statistical Significance | | Significant | | Significant | |

*Statistically Significant Critical Value = 1.7709
*M226 Data removed due to poor replica quality.
**M235 Data removed due to inconsistent facial expression.

TABLE 14
ANALYSIS OF REPLICAS
Shadows East-West

| No. | Panelist No. | Age | Race | Day 0 | Week 4 | % Diff | Week 8 | % Diff |
|---|---|---|---|---|---|---|---|---|
| 1 | M 219 | 59 | C | 21.6 | 29.1 | 34.7 | 26.2 | 21.3 |
| 2 | M 220 | 42 | C | 17.5 | 24.6 | 40.6 | 20.1 | 14.9 |
| 3 | M 221 | 53 | C | 24.4 | 24.0 | −1.6 | 25.7 | 5.3 |
| 4 | M 222 | 49 | C | 28.7 | 22.4 | −22.0 | 29.1 | 1.4 |
| 5 | M 224 | 36 | C | 20.7 | 21.5 | 3.9 | 11.3 | −45.4 |
| 6 | M 227 | 54 | C | 31.5 | 32.9 | 4.4 | 28.7 | −8.9 |
| 7 | M 228 | 61 | C | 28.3 | 20.9 | −26.1 | 29.1 | 2.8 |
| 8 | M 229 | 44 | C | 23.2 | 29.6 | 27.6 | 30.3 | 30.6 |
| 9 | M 230 | 63 | C | 32.0 | 24.9 | −22.2 | 21.0 | −34.4 |
| 10 | M 231 | 45 | A | 32.0 | 31.3 | −2.2 | 33.4 | 4.4 |
| 11 | M 232 | 43 | C | 33.6 | 34.9 | 3.9 | 27.5 | −18.2 |
| 12 | M 233 | 55 | C | 32.5 | 23.5 | −27.7 | 32.9 | 1.2 |
| 13 | M 234 | 59 | C | 20.6 | 24.5 | 18.9 | 32.0 | 55.3 |
| 14 | M 236 | 49 | C | 37.2 | 33.4 | −10.2 | 30.8 | −17.2 |

| | | | | | |
|---|---|---|---|---|---|
| MEAN | 27.41 | 26.96 | 1.57 | 27.01 | 0.94 |
| % Diff | | −1.64 | | −1.48 | |
| t-Stat | | 0.30 | | 0.24 | |
| Statistical Significance | | Significant | | Significant | |

*Statistically Significant Critical Value = 1.7709
*M226 Data removed due to poor replica quality.
**M235 Data removed due to inconsistent facial expression.

TABLE 15
Panelist Questionnaire
4 Week Data
Lab Nos.: M06-2/M06-2A/M06-3
Client No.: H Serum/Night Cream/3Lab Sunblock

| | Strongly | Somewhat | Somewhat | Strongly | Overall |
|---|---|---|---|---|---|
| Statement | Agree | Agree | Disagree | Disagree | Agreement |
| Significantly reduces the | 3 | 5 | 8 | 0 | 50% |
| appearance of fine lines | | | | | |
| and wrinkles | | | | | |
| Significantly reduces | 4 | 6 | 5 | 1 | 63% |
| roughness and dryness | | | | | |
| Significantly diminishes the | 1 | 6 | 9 | 0 | 44% |
| appearance of age spots, | | | | | |
| freckles, and skin | | | | | |
| discolorations | | | | | |
| Significantly lightens the | 1 | 7 | 8 | 0 | 50% |
| skin | | | | | |
| Significantly improves | 6 | 9 | 1 | 0 | 94% |
| skin's softness and | | | | | |
| smoothness | | | | | |
| Significantly improves | 3 | 9 | 4 | 0 | 75% |
| skin's radiance, tone, and | | | | | |
| clarity | | | | | |
| Significantly improves | 4 | 8 | 4 | 0 | 75% |
| skin's firmness, tightness, | | | | | |
| and elasticity | | | | | |
| Significantly moisturizes | 7 | 6 | 3 | 0 | 81% |
| the skin | | | | | |
| Significantly improves | 6 | 6 | 4 | 0 | 75% |
| skin's overall appearance | | | | | |

Note:
1) “Overall Agreement” is expressed as the total number of panelists answering strongly agree and somewhat agree, divided by number of panelists answering the questionnaire (N = 16), multiplied by 100.
2) Data depicted in Agree/Disagree columns are the number of panelists answering the question.

TABLE 16
Panelist Questionnaire
8 Week Data
Lab Nos.: M06-2/M06-2A/M06-3
Client No.: H Serum/Night Cream/3Lab Sunblock

| | Strongly | Somewhat | Somewhat | Strongly | Overall |
|---|---|---|---|---|---|
| Statement | Agree | Agree | Disagree | Disagree | Agreement |
| Significantly reduces the | 6 | 9 | 0 | 1 | 94% |
| appearance of fine lines | | | | | |
| and wrinkles | | | | | |
| Significantly reduces | 11 | 5 | 0 | 0 | 100% |
| roughness and dryness | | | | | |
| Significantly diminishes the | 3 | 9 | 3 | 1 | 75% |
| appearance of age spots, | | | | | |
| freckles, and skin | | | | | |
| discolorations | | | | | |
| Significantly lightens the | 4 | 9 | 2 | 1 | 81% |
| skin | | | | | |
| Significantly improves | 13 | 3 | 0 | 0 | 100% |
| skin's softness and | | | | | |
| smoothness | | | | | |
| Significantly improves | 9 | 4 | 3 | 0 | 81% |
| skin's radiance, tone, and | | | | | |
| clarity | | | | | |
| Significantly improves | 6 | 8 | 2 | 0 | 88% |
| skin's firmness, tightness, | | | | | |
| and elasticity | | | | | |
| Significantly moisturizes | 12 | 3 | 1 | 0 | 94% |
| the skin | | | | | |
| Significantly improves | 9 | 6 | 1 | 0 | 94% |
| skin's overall appearance | | | | | |

Note:
1) “Overall Agreement” is expressed as the total number of panelists answering strongly agree and somewhat agree, divided by number of panelists answering the questionnaire (N = 16), multiplied by 100.
2) Data depicted in Agree/Disagree columns are the number of panelists answering the question.

### Appendix I

### Spectrophotometer Measurements

This instrument utilizes the D/8 geometry conforming to CIE No. 15, ISO 7724/1, ASTM E1164, DIN 5033 Tei17, and JIS Z8722-1982 (diffused illumination/8° viewing system) standards, and offers simultaneous SCI (specular component included) and SCE (specular component excluded) measurements. Light from xenon lamps diffuses on the inner surface of the integrating sphere and illuminates the specimen uniformly. The light reflected by the specimen surface at an angle of 8 degrees to the normal of the surface is received by the specimen-measuring optical system. The diffused light in the integrating sphere is received by the illumination-monitoring optical system and guided to the sensor. The light reflected by the specimen surface and the diffused light are divided into each wavelength component by the specimen-measuring optical system and illumination-monitoring optical sensor, respectively, and then signal proportional to the light intensity of each component are output to the analog processing circuit. By using the outputs from the specimen-measuring optical system and the illumination-monitoring sensor for calculation, compensation for slight fluctuation in spectral characteristics and the intensity of the illumination light is performed. (Double-beam system)

Any increase in the a* value is indicative of a reddening color and a decrease drives the color toward the green shade. An increase in the b* value indicates yellow enhancement and a decrease signifies a color shift into the blue region as is perceived with a blue coefficient. An increase in the L* value indicates lightening of the color and any diminution of the L* value is indicative of darkening of the color.

| | a*-value | b*-value | L*-value |
|---|---|---|---|
| Increase | Reddening | Yellow | Lightening |
| Decrease | Green | Blue | Darkening |

### Appendix II

### Cutometer Measurements

### Explanation of Parameters:

| | |
|---|---|
| R0 | Represents the maximum amplitude on the first curve. The number serves |
| | as an implication of the firmness of the skin. |
| R1 | Represents the minimum amplitude of the first curve. The number indicates |
| | the ability of the skin to return to the original state. |
| R2 | Represents the gross elasticity (ability of redeformation of the skin) of the |
| | skin. The closer to 1 it is the more elastic it is. |
| R3 | Represents the tiring effects on the skin. The comparison of the last |
| | maximum amplitude to the first maximum amplitude, a smaller difference |
| | indicates a smaller tiring effect am litude increases with each new suction). |
| R4 | Represents the tiring effects on the skin. The comparison of the last minimum |
| | amplitude to the first minimum amplitude, a smaller difference indicates a |
| | smaller tiring effect (redeformation decreases with each new suction). |
| R5 | Represents the net elasticity. The closer the value is to 1, the greater the |
| | elasticity. |
| R6 | Represents the viscoelasticity. The smaller the value the higher the elasticity. |
| R7 | Represents the elastic portion of the curve compared to the entire curve. The |
| | closer the value is to 1 the more elastic the curve is. |
| R8 | Represents the amplitude of the relaxation time. The closer the values of R8 |
| | and R0 are too each other the greater the ability of the skin to return to its |
| | original state. |
| R9 | Represents the tiring of the skin after repeatedly being suctioned in. The |
| | smaller the value of R9 the smaller the tiring effects. |
| F0 | Represents the maximum amplitude multiplied by the suction time. The closer |
| | the value is to 0 the more elastic the skin is. |
| F1 | Represents the maximum amplitude multiplied by the relaxation time. The |
| | closer the value is to 0 the more elastic the skin is. |
| F4 | Represents the area within and above the envelope curve. The smaller the |
| | value the firmer the skin. F4 utilizes the calculations for F2 and F3. |

The invention is not limited by the embodiments described above which are presented as examples only but can be modified in various ways within the scope of protection defined by the appended patent claims.

We claim:

1. A cosmetic anti-aging composition comprising an effective amount of Human Oligopeptide-9 and an effective amount of at least one additional oligopeptide.

2. The cosmetic composition of claim 1 wherein the at least one additional oligopeptide is selected from the group consisting of Oligopeptide-4 and Oligopeptide-5.

3. The cosmetic composition of claim 1 wherein the at least one additional oligopeptide is Oligopeptide-5.

4. The cosmetic composition of claim 1 wherein the amount of Human Oligopeptide-9 is from about 0.001% to about 1% based on the total weight of the cosmetic composition.

5. The cosmetic composition of claim 1 wherein the amount of at least one additional oligopeptide is from about 0.001% to about 1% based on the total weight of the cosmetic composition.

6. The cosmetic composition of claim 1 wherein the ratio of Human Oligopeptide-9 and the at least one additional oligopeptide by weight is in the range of about 1:1 to about 1:5.

7. The cosmetic composition of claim 1 further comprising at least one other cosmetically acceptable ingredient.

8. The cosmetic composition of claim 7 wherein the other cosmetically acceptable ingredient is selected from the group consisting of skin brightening agents, antioxidants (free radical scavengers), botanical extracts, emulsion stabilizers, thickening agents, emulsifiers, emollients, solvent, skin softener, and fragrance.

9. The cosmetic composition of claim 1 further comprising water, disodium EDTA, xanthan gum, butylene glycol, propyl gallate, glycerin, dimethyl isosorbide, hydroxyethylcellulose, polymethylmethacrylate, C10-18 triglyceride, octyldodecyl behenate, neopentyl glycol dicaprate, PPG-3 benzyl ether myristate, dimethicone, C12-20 acid PEG-8 ester, cetearyl glucoside, steareth-2, tocopheryl acetate, hydroxyethyl acrylate/sodium acryloyidimethyl taurate copolymer, squalane, polysorbate 60, diacetyl boldine, lecithin, EDTA, xylitylglucoside, anhydroxylitol, xylitol, Sodium Hyaluronate, Ginko Biloba leaf extract, butylene glycol, Beta Vulgaris (Beet) root extract, yeast extract, Camellia Sinensis (Green tea) leaf extract, hydrolyzed glycosaminoglycans, glycerin, sodium levulinate, citric acid, and fragrance.

10. The cosmetic composition of claim 1 is in a form selected from the group consisting of serum, cream, lotion, and gel, preferably serum.

11. The cosmetic composition of claim 1 is in a form of serum.

12. The cosmetic composition of claim 1 having a pH range of about 4 to 7.

13. The cosmetic composition of claim 1 having pH range of about 4 to 6.

14. The cosmetic composition of claim 1 having pH range of about 4.5 to 5.

15. The cosmetic composition of claim 1 further comprising an effective amount of hydrolyzed glycosaminoglycans, xylitol derivatives, and a blend of Beet root extract, Haberlea Rhodopensis leaf extract and yeast extract.

16. The cosmetic composition of claim 15 wherein the amount of hydrolyzed glycosaminoglycans is from about 0.1% to about 1% based on the total weight of the cosmetic composition.

17. The cosmetic composition of claim 15 wherein the amount of xylitol derivatives is from about 0.1% to about 3% based on the total weight of the cosmetic composition.

18. The cosmetic composition of claim 15 wherein the total amount of the blend mixture of Beet root extract, Haberlea Rhodopensis leaf extract and yeast extract is from about 0.1 to about 3% based on the total weight of the cosmetic composition.

19. The cosmetic composition of claim 15 wherein the ratio of hydrolyzed glycosaminoglycans and the xylitol derivatives by weight is in the range of 1:1 to 1:5.

20. The cosmetic composition of claim 15 wherein the ratio of hydrolyzed glycosaminoglycans and the blend mixture of Beet root extract, Haberlea Rhodopensis leaf extract and yeast extract by weight is in the range of 1:1 to 1:5.

21. A cosmetic composition comprising an effective amount of hydrolyzed glycosaminoglycans, xylitol derivatives, and a blend of Beet root extract, Haberlea Rhodopensis leaf extract, and yeast extract.
